SEQUENCE LISTING (6 sequences)

SEQ ID NO: 1 - protein, 8 aa, Artificial
  epitope
SIINFEKL

SEQ ID NO: 2 - protein, 9 aa, Artificial
  epitope
SYIGSINNI

SEQ ID NO: 3 - DNA, 36 bp, Artificial
  primer
atttaggtgacactatagagtagttcgcctgtgtga

SEQ ID NO: 4 - DNA, 33 bp, Artificial
  primer
gaggagatctaagcatgcacgttcacggagaga

SEQ ID NO: 5 - DNA, 29 bp, Artificial
  primer
ggctctagaccatgtctaagaaaccagga

SEQ ID NO: 6 - DNA, 32 bp, Artificial
  primer
gaggagatctaagcatgccgttcacggagaga

(19) United States
(12) Patent Application Publication
(10) Pub. No.: US 2006/0280757 A1
(43) Pub. Date: Dec. 14, 2006

(54) Flavivirus vaccine delivery system

(76) Inventor: Alexander A. Khromykh, The Gap (AU)

Correspondence Address: INTELLECTUAL PROPERTY GROUP;FREDRIKSON & BYRON, P.A., 200 SOUTH SIXTH STREET, SUITE 4000, MINNEAPOLIS, MN, 55402, US

(21) Appl. No.: 10/559,146

(22) PCT Filed: Jun. 7, 2004
(86) PCT No.: PCT/AU04/00752

§ 371 Date: May 26, 2006

(30) Foreign Application Priority Data
Jun. 6, 2003 (AU) 2003902842

(51) Int. Cl. A61K 39/12; C12N 7/00; C12N 15/86; A61K 39/193; C12N 5/06

(52) U.S. Cl. 424/218.1; 435/235.1; 435/456; 435/325; 977/802

(57) ABSTRACT

A tetracycline regulatable flaviviral packaging system is provided that facilitates expression of flaviviral structural proteins necessary for flaviviral RNA replicon packaging and virus like particle production in animal cells. This regulatable packaging system is compatible with Kunjin, Dengue and West Nile virus and other flaviviral replicon-based expression systems and produces unexpectedly high titres of virus-like particles. A particular application of this packaging system is the production of virus-like particles that package RNA comprising a flaviviral replicon and encoding a heterologous protein or peptide for expression in animal cells. Even more particularly, the packaging system is capable of delivering immunogens that induce a protective CD8 T cell-mediated immune response.

### FIELD OF THE INVENTION

THIS INVENTION relates to production of virus-like particles of flaviviral origin. More particularly, this invention relates to an inducible flaviviral packaging system that facilitates inducible expression of flaviviral structural proteins necessary for flaviviral RNA packaging in animal cells. In a particular form, the invention provides a tetracycline-inducible packaging system compatible with Kunjin and other flaviviral expression systems that produces unexpectedly high titres of virus-like particles. A particular application of the packaging system is the production of virus-like particles that package RNA comprising a flaviviral replicon and encoding a heterologous protein or peptide for expression in animal cells.

### BACKGROUND OF THE INVENTION

Replicon-based vectors of positive strand RNA viruses have been developed for anti-viral and anti-cancer vaccines (reviewed in Khromykh, 2000. Curr Opin Mol Ther. 2:555-569). Several features make these vectors a desirable choice for development of highly efficient and safe vaccines. These include: (i) high level of expression of encoded heterologous genes (HGs) due to the ability of replicon RNA to amplify itself, (ii) exclusively cytoplasmic replication which eliminates any possible complications associated with nuclear splicing and/or chromosomal integration, (iii) inability of the replicon RNA to escape from transfected (or infected) cell thus limiting the spread of the vaccine vector in the immunized subject which makes these vectors biologically safe, and (iv) relatively small genome size (7-9 kb) allowing easy manipulations with their cDNA and generation of recombinants.

Replicon-based expression vectors have been developed for representatives of most positive strand RNA virus families, including alphaviruses, picornaviruses, and flaviviruses (reviewed in Khromykh, 2000 supra).

In general, VLP delivery has shown to be the most efficient in terms of inducing protective immune responses in mammals.

In particular, expression systems utilizing Kunjin (KUN) flaviviral VLPs have been shown to induce protective immune responses to viral proteins, as described in International Application PCT/AU02/01598.

However, packaging of KUN replicon RNA into VLPs is relatively elaborative and time consuming and requires two consecutive transfections, first with KUN replicon RNA and after a 24-36 hr delay with the SFV replicon, RNA expressing KUN structural genes (Khromykh, et al., 1998. J Virol. 72 5967-5977) In addition, the maximum titres of VLPs produced using this system were only about 2 to 5×10⁶ infectious VLPs per ml (Khromykh et al., 1998, supra; Varnavski & Khromykh, 1999, Virology. 255 366-375) which makes large scale VLP manufacture difficult and inefficient.

Flavivirus structural proteins appear to be one of the primary causes of viral cytopathicity and virus-induced apoptosis (Nunes Duarte dos Santos et al., 2000. Virology 274 292-308). Low cytopathicity of flavivirus replicons compared to the full-length RNA (1, 2, 4, 9-11, 13, 14) also demonstrates the major contribution of structural proteins to viral cytopathicity. Although stable cell lines expressing a prM and E cassette from DEN2 and JE viruses have been generated, the expression levels were low when the native prM-E genes were used (Hunt et al., 2001, J. Virol. Methods. 97 133-149). Inactivation of the furin cleavage site in prM protein to produce immature prM-E particles with low fusogenic activity (Konishi et al., J Virol. 75 2204-2212), or co-expression of anti-apoptotic bcl-2 gene (Konishi & Fujii, 2002, Vaccine. 20 1058-1067), was required to establish stable cell lines expressing relatively high amounts of prM-E particles. None of these stable cell lines simultaneously expressed all three flavivirus structural proteins.

Previous attempts by the present inventors to generate a stable cell line continuously expressing all three KUN structural genes under control of separate promoters (expressing C and prM-E separately), resulted in great instability of expression, producing only 10-20% positively expressing cells after a few cell passages. Attempts to use these cell lines to produce KUN replicon VLPs resulted in very low VLP titres

### OBJECT OF THE INVENTION

It is therefore an object of the invention to provide a flavivirus packaging system that achieves more efficient and/or higher yield VLP production than prior art packaging systems.

### SUMMARY OF THE INVENTION

The invention is therefore broadly directed to a regulatable flavivirus packaging system, packaging construct and/or packaging cell comprising same.

Although International Publication WO 99/28487 briefly mentions that establishment of a cell line that stably and inducibly expresses flavivirus structural proteins would be a useful approach for the production of VLPs, the present inventors have surprisingly found that inducible expression of C and prM-E is not in itself sufficient to enable high yield and high efficiency VLP packaging.

In undertaking the establishment and practical implementation of a regulatable flavivirus packaging system, the present inventors have unexpectedly shown that structural proteins C, prM and E must be expressed as a single, precursor translation product rather than as separate C and prM-E proteins to produce much higher amounts of VLPs than might have been expected from the prior art.

A particular advantage of the present invention is that VLP titres are at least 500-fold greater than titres typically obtained using prior art packaging systems.

Another particular advantage of the present invention is that the regulatable flavivirus packaging system may be useful for packaging replicons derived from any of a variety of flavivirus subgroups.

In a first aspect, the invention provides a packaging construct for regulatable expression of flavivirus structural proteins in an animal cell, said vector comprising a regulatable promoter operably linked to a nucleotide sequence encoding a flavivirus structural protein translation product which comprises C protein, prM protein and E protein.

In a second aspect, the invention provides a packaging cell comprising the packaging construct of the first-mentioned aspect.

In a third aspect, the invention provides a flaviviral expression system comprising:

(i) a packaging construct for regulatable expression of flavivirus structural proteins in an animal cell, said vector comprising a regulatable promoter operably linked to a nucleotide sequence encoding flavivirus structural proteins; and

(ii) a flaviviral expression construct comprising:
(a) a flaviviral replicon;
(b) a heterologous nucleic acid; and
(c) a promoter operably linked to said replicon.

Preferably, according to the aforementioned aspects the regulatable promoter is tetracycline inducible.

In a fourth aspect, the invention provides a packaging cell comprising the flaviviral expression system of the invention.

In a fifth aspect, the invention provides a method of producing flavivirus VLPs including the step of:

(i) introducing the packaging construct of the first aspect into a host cell to thereby produce a packaging cell;

(ii) introducing into said packaging cell a flaviviral expression construct comprising:
(a) a flaviviral replicon;
(b) a heterologous nucleic acid; and
(c) a promoter operably linked to said replicon; and

(iii) inducing production of one or more VLPs by said packaging cell.

In a sixth aspect, the invention provides flavivirus VLPs produced according to the method of the fifth aspect.

In a seventh aspect, the invention provides a pharmaceutical composition comprising the VLPs of the sixth aspect and a pharmaceutically acceptable carrier diluent or excipient.

In an eighth aspect, the invention provides a method of producing a recombinant protein including the step of infecting a host cell with the VLPs of the sixth aspect, whereby said heterologous nucleic acid encoding said protein is expressed in said host cell.

Suitably, the expressed protein is subsequently purified.

In a ninth aspect, the invention provides a method of immunizing an animal including the step of administering the pharmaceutical composition of the seventh to the animal to thereby induce an immune response in the animal.

Preferably, the animal is a mammal.

More preferably, the mammal is a human.

Preferably, according to the aforementioned aspects the C, prM, and E structural proteins are of Kunjin virus (KUN) origin.

Preferably, according to the aforementioned aspects the flaviviral replicon is of Kunjin virus, West Nile virus or Dengue virus origin.

In particular embodiments, the flaviviral replicon encodes one or more mutated non-structural proteins.

Throughout this specification, unless otherwise indicated, “comprise”, “comprises” and “comprising” are used inclusively rather than exclusively, so that a stated integer or group of integers may include one or more other non-stated integers or groups of integers.

### BRIEF DESCRIPTION OF THE FIGURES

FIG. 1. Generation and characterization of stable packaging cell line tetKUNCprME. (A) Schematic representation of the plasmid constructs used for generation of stable packaging cell line tetKUNCprME. pEF-tTA-IRESpuro plasmid was used to generate a first stable BHK cell line, BHK-Tet-Off, continuously expressing the tetracycline transactivator (tTA) from the human elongation factor 1α promoter (pEF-1a). tetKUNCprME, expressing KUN structural genes C, prM, and E (KUN CprME) from tetracycline-inducible CMV promoter (P_{minCMV}) was established by transfection of pTRE2 CprME-IRESNeo plasmid DNA into BHK-Tet-Off cells and selection or cells growing in the presence of G418 and puromycin (see text). In uninduced tetKUNCprME cells doxycycline (DOX; a form of tetracycline with higher specific activity) binds to tTA and prevents it from binding to the tetracycline responsive element (TRE) and subsequent activation of CprME mRNA transcription from CMV promoter. To induce expression of KUN CprME genes, DOX is removed from the medium resulting in the release of tTA, its binding to TRE, and activation of CprME mRNA transcription from CMV promoter. tetR—Tet repressor protein; VP16—Herpes simplex virus VP16 activation domain; IRES—EMCV internal ribosome entry site; puro—puromycin N-acetyl transferase; TRE—Tetracycline-response element; Neo—neomycin resistance gene; SV40 polyA—SV40 transcription terminator/poly(A) signal; β-globin polyA—β-globin transcription terminator/poly(A) signal. (B) Production of secreted E protein and VLPs in induced and uninduced tetKUNCprME cells in the presence and absence of KUN replicon RNA. “−RNA” graph (left part) shows the results of an experiment without replicon RNA transfection, “+RNA” graph (right part) shows the results of another experiment with electroporation of KUN replicon RNA—RNAleu. tetKUNCprME cells either electroporated with KUN replicon RNA (“+RNA”) or not electroporated (“−RNA”) and maintained for 48 h in the medium with (uninduced) or without (induced) 0.5 ug/ml of doxycycline. Detection of secreted KUN E protein (white bars) by antigen capture ELISA and determination of VLP titres (black bars) (in infectious units (IU) per ml) by infectivity assay on Vero cells were performed as described in Materials and Methods. Negative controls in both experiments (Cont) were culture fluids from normal BHK cells. The titres of KUN virus positive controls (KUN) used in each experiment were determined by plaque assay on BHK cells.

FIG. 2. Schematic overview of processing of Kunjin virus structural proteins C, prM and E. Cleavage sites are indicated as: □ NS2B-NS3 (viral) Protease; ●Host Cell Signalase; ←Host cell furin protease.

FIG. 3. Induction of KUN structural gene expression in tetKUNCprME cells upon removal of doxycycline. (A) Northern blot hybridisation analysis of RNA extracted from induced (−DOX) and uninduced (+DOX) tetKUNCprME and BHK cells. 20 μg of each RNA was separated on a 1% formamide-agarose gel then transferred onto Hybond N membrane by capillary blotting. (B) Western blot analysis of protein extracted from induced (−DOX) and uninduced (+DOX) tetKUNCprME and BHK cells. 5 μg of total protein was separated on a 12.5% polyacrylamide gel then transferred onto Hybond P membrane. The membrane was incubated with KUN anti-E monoclonal antibodies and bound KUN E protein was detected by chemiluminescence.

FIG. 4. Amplification and spread of KUN replicon VLPs in tetKUNCprME cells. Coverslips of tetKUNCprME and BHK21 cells were infected with 0.1 MOI (Multiplicity of Infection) of RNAleuMpt VLPs and analysed by IF with KUN anti-NS3 antibodies at 2d and 3d after infection.

FIG. 5. CD8 T cell responses in mice immunised with high titre KUN VLP replicons. (A) C57BL/6 mice (n=4 per group) were immunised intraperitoneally with PBS (Naive), 10⁸ IU of KUN VLPs not encoding a recombinant antigen (KUN VLP Control), or the indicated dose of KUN VLPs encoding the murine polytope KUN-Mpt VLP (Anraku et al.,. 2002, J Virol. 76 3791-3799). After 2 weeks splenocytes were removed and analysed for (H-2 Kb restricted) SIINFEKL-specific responses by IFNγ ELISPOT. (B, C) BALB/c mice (n=3 per group) were immunised intraperitoneally with 2.5×10⁷ μl of KUN VLPs encoding respiratory syncytial virus matrix 2 protein (KUN-M2 VLP), 2.5×10⁷ IU of KUN VLP not encoding a recombinant antigen (KUN VLP Control), or subcutaneously with a peptide vaccine containing the H-2 Kd restricted RSV M2 epitope, SYIGSINNI, formulated with tetanus toxoid in Montanide ISA 720 (SYIGSINNI/TT/M720) as described previously (Elliott et al., 1999, Vaccine. 17 2009-2019) After 2 weeks splenocytes were removed and analysed for SYIGSINNI-specific responses by (B) IFNγ ELISPOT and (C) by standard chromium release assay (black squares—P815 target cells sensitised with SYIGSINNI peptide, white squares—P815 target cells without peptide) as described previously (Anraku et al., 2002, supra).

FIG. 6. Tumour therapy with KUN VLP and IL-2. Four groups of mice were injected with 5×104 LLOva by the s.c. route on the back. Once the LLOva tumours were palpable (>1 mm2), mice were vaccinated with KUN VLPMpt or PBS (Control) 2 times, with and without IL-2 at the times indicated on the graph. (A) Tumour size was monitored and groups compared by ANOVA; VLP vs VLP+IL-2, p=0.34; Control vs Control+IL-2, p=0.96; Control vs VLP, p<0.001; Control vs VLP+IL-2, p<0.001. (B) Survival represented in a Kaplan Meier plot for the same experiment, (animals were euthanased when 1 tumour reached 15×15 mm2). Groups compared by Log Rank statistic; VLP vs VLP+IL-2, p=0.11; Control vs Control+IL-2, p=0.41; Control vs VLP, p=0.0015; Control vs VLP+IL-2, p<0.001.

FIG. 7. Adaptive mutations confer advantage in establishing persistent replication of KUN replicon RNA in BHK21, HEp-2 and 293 cells after infection with replicon VLPs. BHK21, HEK293 and HEp-2 cells were infected with wild type rep/PAC-βgal replicon VLPs or each of the NS2A mutants at MOI of 0.01, 1 and 10, respectively. At 48 hours post-infection 1 μg/ml (HEK293 and HEp-2) and 5 μg/ml (BHK21) of puromycin were added to the medium and cells were propagated for an additional 7 days. Puromycin-resistant cell colonies were fixed in 4% formaldehyde and stained either with crystal violet (BHK21) or with X-gal (HEK293 and HEp-2).

FIG. 8. The use of tetKUNCprME cells for enhanced expression of heterologous genes from Kunjin replicon vector. KUN packaging A8 cell line and BHK21 cells in 24 well plate at 95% confluent were infected with KUN repPAC/β-gal VLPs at MOI=1 and analyzed by X-gal staining (A) and β-gal assay (B) at 2 4, and 6 days after infection. The blank bar represent of BHK21 cells and the filled Bar represents of KUN packaging of A8 cells. Each bar represents average value from duplicate samples. The error bars represent standard deviation.

### DETAILED DESCRIPTION OF THE INVENTION

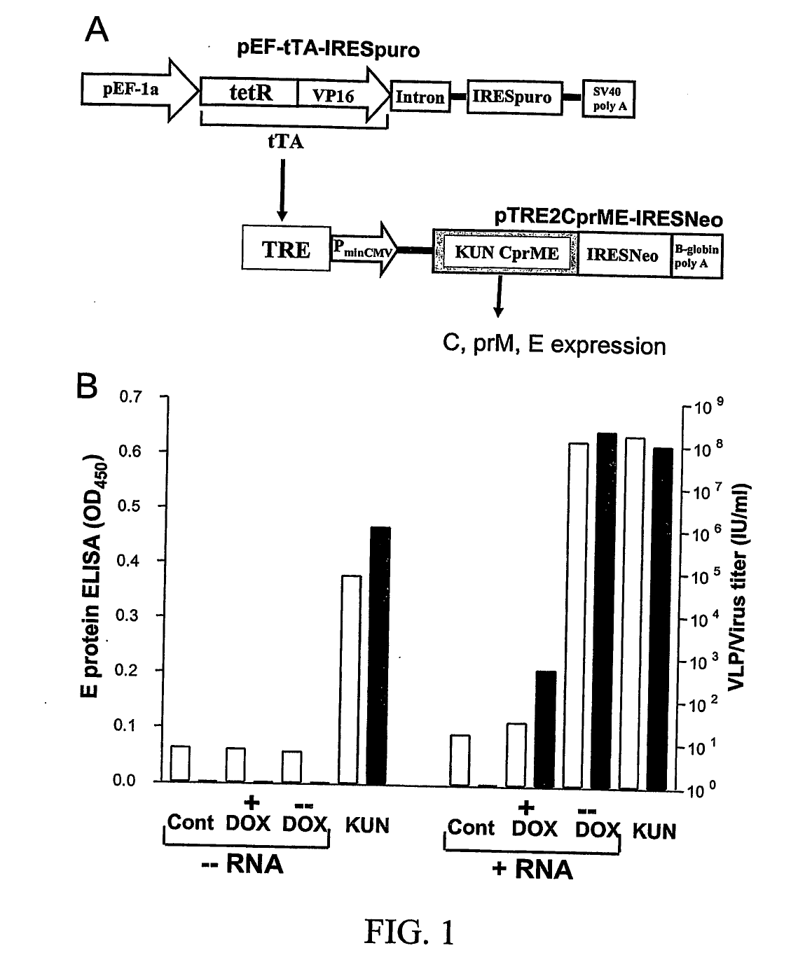

The present inventors have developed a stable packaging construct and packaging cell line tetKUN-CprME that allows simplified (i.e one RNA transfection) inducible manufacture of KUN replicon VLPs. In the stable packaging cell line of the invention, KUN structural genes C, prM and E are expressed from the tetracycline-inducible CMV promoter (FIG. 1). During propagation and maintenance of this packaging cell line production of toxic KUN structural gene products is inhibited by addition of tetracycline (or doxycycline) to the medium. The removal of doxycycline from the medium after transfection of KUN replicon RNA into tetKUN-CME cells results in induction of KUN structural genes expression whose products then package replicating KUN replicon RNA into secreted VLPs (FIG. 1).

Surprisingly, KUN structural proteins produced from this packaging construct of the invention were capable of packaging transfected and self-amplified Kunjin replicon RNA into secreted VLPs at titres of up to ˜10⁹ VLPs per ml. This represents ˜1500 fold improvement over previous packaging protocol employing cytopathic Semliki Forest virus replicon RNA for transient expression of Kunjin structural genes. Secreted KUN replicon VLPs could be harvested continuously three to four times for up to eight days after RNA transfection producing a total amount of up to ˜5.4×10¹⁰ VLPs from 3×10⁶ transfected cells (Table 3). Passaging of VLPs on Vero cells and intracerebral injection of VLPs into 2-4 days old suckling mice showed no evidence for the presence of any infectious Kunjin virus in VLP preparations. Immunization of mice with KUN replicon VLPs encoding human respiratory syncytial virus M2 gene induced exceptionally strong CD8+ T cell responses. Packaging cells were also capable of packaging replicon RNA from a distantly related Flavivirus, dengue virus type 2 as well as West Nile virus replicon, indicating potential for these cells to package any flavivirus replicon RNA.

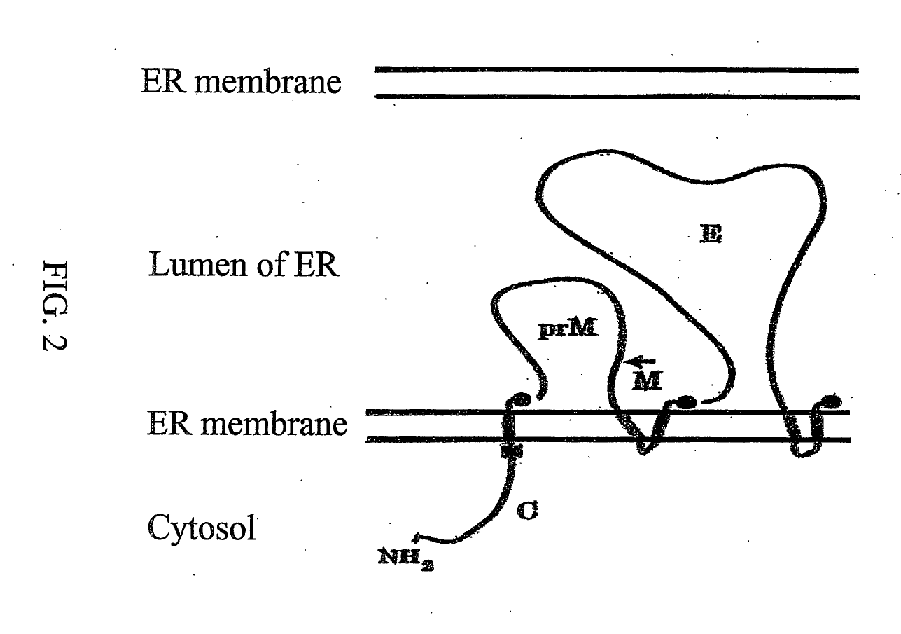

Processing of flavivirus structural protein from a C-prM-E precursor translation product to individual C, prM(M) and E proteins that are required to produce virus particles (or replicon VLPs) is a complicated process and requires five cleavage events by cell signalase, cell furin protease and virus-encoded protease NS2B-NS3 (FIG. 2). The contiguous C-prM-E precursor translation product employed according to the present invention thus cannot be processed correctly without supplying viral protease expressed from replicon RNA. This is indicated in FIG. 1, which shows that no secreted E protein (an indicator of secreted VLPs) was produced from tetKUNCprME cells upon induction of C-prM-E expression unless the cells were transfected with replicon RNA. The cleavage of native flavivirus C-prM junction requires cleavage by both viral and cell protease and unless viral protease cleavage has occurred, cell signalase cleavage can not proceed (Stocks & Lobigs, 1998, J Virol. 72 2141-2149). This leads to accumulation of uncleaved C-prM product in the ER that may trigger ER stress response detrimental for cell. In addition, if prM is not cleaved from C it cannot participate in formation of prM-E heterodimer that is essential for production of secreted virus particles. Although mutations in the hydrophobic sequence between C and prM allowing efficient cleavage of prM from C by cell signalase without viral protease can be designed they appear to abolish production of virus particles (Lee et al., 2000, J Virol. 74 24-32.), suggesting an important role for co-ordinated processing of C-prM junction by cell and viral proteases for production of secreted virus particles. Thus it is likely that employed in this invention expression of a nucleotide sequence encoding a C-prM-E precursor translation product in conjunction with transfection of replicon RNA that encodes viral protease provided conditions favourable for proper processing of KUN structural proteins and production of high titres of secreted replicon VLPs.

More particularly, the inducible expression system of the present invention provides an ability to “switch off” the expression of the potentially toxic C-prM-E precursor translation product by addition of tetracycline to the cell culture medium. This allows selection and maintenance of tetKUNCprME stable packaging cell line without decreasing C-prM-E expression and hence allows high level, inducible production of high titres of replicon VLPs.

It will be appreciated that the present invention may therefore have the following broad applications to flavivirus replicon packaging:

(i) an ability to package any flavivirus replicon; and/or

(ii) an ability to express any flavivirus structural proteins necessary and sufficient for flaviviral replicon packaging

As used herein, “flavivirus” and “flaviviral” refer to members of the genus Flavivirus within the family Flaviviridae, which contains 65 or more related viral species. Typically, flavivirus are small, enveloped RNA viruses (diameter about 45 nm) with peplomers comprising a single glycoprotein E. Other structural proteins are designated C (core) and M (membrane-like). The single stranded RNA is infectious and typically has a molecular weight of about 4×10⁶ with an m7G ‘cap’ at the 5′ end but no poly(A) tract at the 3′ end; it functions as the sole messenger. Flaviviruses infect a wide range of vertebrates, and many are transmitted by arthropods such as ticks and mosquitoes, although a separate group of flaviviruses is designated as having no-known-vector (NKV).

Particular, non-limiting examples of flavivirus are West Nile virus, Kunjin virus, Yellow Fever virus, Japanese Encephalitis virus, Dengue virus, Tick-borne encephalitis, Murray Valley encephalitis, Sent Louis encephalitis, Montana Myotis leukoencephalitis virus, Usutu virus, and Alkhurma virus.

The term “nucleic acid” as used herein designates single-or double-stranded mRNA, RNA, cRNA, RNA-DNA hybrids and DNA inclusive of cDNA and genomic DNA.

In a preferred form, the packaging construct of the invention is a double-stranded plasmid DNA packaging construct.

By “protein” is meant an amino acid polymer. Amino acids may include natural (i.e genetically encoded), non-natural, D- and L-amino acids as are well known in the art.

A “peptide” is a protein having less than fifty (50) amino acids.

A “polypeptide” is a protein having fifty (50) or more amino acids.

According to the present invention, a “packaging construct” comprises a regulatable promoter operably linked to one or more nucleotide sequences encoding one or more flaviviral structural proteins.

Suitably, the packaging construct comprises a nucleotide sequence encoding structural proteins C, prM and E.

It has been found by the present inventors that inducible expression of a contiguous amino acid sequence encoding C, prM and E structural proteins as a “precursor” or “pre-protein” is by far the most efficacious system for producing VLPs. This is in contrast to typical prior art approaches where C and prM-E proteins are respectively encoded by separate nucleotide sequences.

In this regard, according to the invention the structural proteins C, prM and E are expressible in an animal cell as a single, precursor translation product which can undergo subsequent proteolytic processing to produce individual C, prM and E structural proteins required for VLP production.

A proposed model that describes processing of the precursor translation product is summarized in FIG. 2.

Although processing normally relies upon the presence of both cellular proteases and replicon-encoded proteases, it is also contemplated that alternative protease cleavage sites could be engineered into one or more of the structural proteins C, prM and E which, together with expression of appropriate proteases by the animal host animal cell, could provide an alternative processing system to that which normally occurs.

Such a system could abrogate the requirement for flaviviral replicon-encoded proteases in processing of the C, prM and E translation product.

In a preferred embodiment, the structural proteins are the KUN structural proteins C, prM and E.

However, structural proteins from any other flavivirus may be used. It is well established that replacement of structural proteins in one flavivirus with those of another or other flaviviruses permits recovery of chimeric flaviviruses (Monath et al., 2000, J. Virol. 74 1742; Guirakhoo et al., 2000, J. Virol. 74 5477; Pletnev et al., 1992, Proc. Natl. Acad. Sci. USA 89 10532) demonstrating that structural proteins from one flavivirus are capable of packaging RNA from another flavivirus. It has recently been shown that (i) yellow fever replicons can be packaged by providing yellow fever prME and West Nile or Dengue virus core proteins, and (ii) that West Nile replicons can be packaged by providing virus.

It will also be appreciated that structural proteins C, prM and E include and encompass any mutations or other sequence variations in one or more of these proteins that do not prevent, or do not appreciably diminish, processing of the C, prM and E translation product and/or viral packaging.

In this regard, reference is made to the aforementioned possibility that alternative protease cleavage sites could be engineered into one or more of the structural proteins C, prM and E. In addition, sequences directly upstream or downstream of the cleavages sites recognised by viral and cellular proteases can be modified to enhance cleavage efficiency (Stocks & Lobigs et al., 1998, J Virol, 72 2141-2149) which may lead to improved cleavage and/or secretion of VLPs.

Typically, it is contemplated that mutated and/or variant structural proteins may have at least 80%, preferably at least 85%, more preferably at least 90% or advantageously at least 95%, 96%, 97%, 98% or 99% amino acid sequence identity with a C, prM or E protein amino acid sequence respectively.

Accordingly, it will be appreciated that a nucleotide sequence encoding a mutated and/or variant structural proteins may have at least 70%, preferably at least 75%, more preferably at least 80%, even more preferably at least 90% or advantageously at least 95%, 96%, 97%, 98% or 99% nucleotide sequence identity with a nucleotide sequence encoding C, prM or E protein.

“Percent sequence identity” as used herein is a percentage determined by the number of exact matches of amino acids or nucleotides to a reference sequence divided by the number of residues in the region of overlap. A minimum region of overlap is typically at least 6, 12 or 20 contiguous residues. Amino acid sequence identity may be determined by standard methodologies, including the NCBI BLAST search methodology available at www.ncbi.nlm.nih.gov, inclusive of non-gapped BLAST and Gapped Blast 2.0. However, sequence analysis methodologies described in U.S. Pat. No. 5,691,179 and Altschul et al., 1997, Nucleic Acids Res. 25 3389-3402 are also contemplated.

A feature of the packaging construct of the present invention is the presence of a regulatable promoter operably linked to the nucleotide sequence encoding a flavivirus structural protein translation product.

By “regulatable promoter” is meant any promoter operable in an animal cell, wherein promoter activity is controllable in response to one or more regulatory agents. Regulatory agents may be physical (e.g. temperature) or may be chemical (e.g. steroid hormones, heavy metals, antibiotics).

Examples of such promoters include heat-shock inducible promoters, ecdysone inducible-promoters, tetracycline-inducible/repressible promoters, metallothionine-inducible promoters and mammalian-operable promoters inducible through the bacterial lac operon (e.g. lac-regulated CMV or RSV promoter).

A preferred regulatable promoter is a “tet off” promoter which is repressed in the presence of doxycylcine and induced by removal of doxycycline.

According to a particularly preferred form of this embodiment, the regulatable promoter comprises a CMV promoter linked to a tetracycline response element (TRE) that facilitates responsiveness to a tetracycline transactivator (tTA) encoded by a separate construct.

The packaging construct of the invention may further comprise other regulatory sequences such as an internal ribosomal entry site (IRES), 3′ polyadenylation and transcription terminator sequence (e.g. β-globin or SV40-derived) and a selectable marker gene (e.g. neomycin, hygromycin or puromycin resistance genes) to facilitate selection of stable transformants.

In a particularly preferred form, the packaging construct of the invention comprises an IRES—neomycin nucleotide sequence to facilitate selection of stable transfectants.

In a preferred form of this embodiment, the packaging construct further comprises a β-globin polyadenylation signal.

According to the invention, a stable packaging cell line is typically developed in two stages:

(i) establishment of a stable cell line expressing tetracycline (doxycycline) transactivator; and

(ii) use of the stable cell line produced in (i) to generate a packaging cell capable of inducibly expressing KUN structural genes after withdrawal of doxycycline.

In a particular embodiment, the stable cell line at step (i) is produced by transfecting into the cell a tetracycline transactivator construct comprising a tetracycline transactivator nucleotide sequence operably linked to a human elongation factor α promoter. However, it will be appreciated that other promoters may be useful in this regard, such as RSV, SV40, alpha crystallin, adenoviral and CMV promoters, although without limitation thereto.

By “operably linked” or “operably connected” is meant that said regulatable promoter is positioned to initiate and regulatably control intracellular transcription of RNA encoding said flaviviral structural proteins.

Preferably, the tetracycline transactivator construct further comprises an IRES puromycin selection marker sequence that facilitates selection of stable transfectants.

At step (ii), a packaging construct of the invention as hereinbefore described is then transfected into the tetracycline transactivator-expressing stable cell line.

Suitable host cells for VLP packaging may be any eukaryotic, animal or mammalian cell line that is competent to effect transcription, translation and any post-transcriptional and/or post-translational processing or modification required for protein expression. Examples of mammalian cells typically used for nucleic acid transfection and protein expression are COS, Vero, CV-1, BHK21, 293, HEK, Chinese Hamster Ovary (CHO) cells, NIH 3T3, Jurkat, WEHI 231, HeLa MRC-5, and B16 melanoma cells without limitation thereto.

Preferably, the host cell is BHK21.

It will be appreciated that packaging cells produced according to the invention may be used for subsequent packaging of flaviviral replicon RNAs encoding one or more proteins.

Flavivirus replicons contemplated by the present invention include any self-replicating component(s) derivable from flavivirus RNA as described for example in International Publication WO 99/28487 and International Application 02/01598. These include without limitation herein DNA-based replicon constructs where replicon cDNA is placed under the control of a mammalian expression promoters such as CMV and delivered in a form of plasmid DNA, and RNA-based replicon constructs where replicon cDNA is placed under the control of a bacteriophage RNA polymerase promoter such as SP6, T7, T3 that allows production of replicon RNA in vitro using corresponding DNA-dependent RNA polymerases and where said replicon RNA can be delivered as naked RNA or as RNA packaged into VLPs.

Although a preferred flaviviral replicon of the invention is derived from Kunjin virus, it will be appreciated by persons skilled in the art that the packaging system of the present invention may be used for packaging any flaviviral replicon.

Examples of flavivirus replicons that are relatively well characterized include replicons from West Nile Virus strains of lineage 1 (Shi et al., Virology, 2002, 296 219-233) and lineage II (Yamshchikov et al., 2001, Virology, 281 294-304), dengue virus type 2 (Pang et al., 2001, BMC Microbiology, 1 18), and yellow fever virus (Molenkamp et al., 2003, J. Virol., 77 1644-1648).

In one particular embodiment, said flaviviral replicon may encode one or more mutated structural proteins inclusive of NS1, NS2A, NS2B, NS3, NS4A, NS4B and/or NS5.

In one particular embodiment, leucine residue 250 of the NS1 protein is substituted by proline.

In another particular embodiment, Alanine 30 is substituted by Proline in the nonstructural protein NS2A.

In yet another particular embodiment, Asparagine 101 is substituted by Aspartate in the nonstructural protein NS2A.

In still yet another particular embodiment, Proline 270 is substituted by Serine in the nonstructural protein NS5.

It will also be appreciated that alternative amino acids may be used to those described above to thereby introduce cell-adaptive mutations into the replicon.

According to the present invention a “flaviviral expression vector” comprises a flavivirus replicon together with one or more other regulatory nucleotide sequences. Such regulatory sequences include but are not limited to a promoter, internal ribosomal entry site (IRES), restriction enzyme site(s) for insertion of one or more heterologous nucleic acid(s), polyadenylation sequences and other sequences such as an antigenomic sequence of the hepatitis delta virus ribozyme (HDVr) that ensure termination of transcription and precise cleavage of 3′ termini, respectively.

In a particularly preferred form, the flaviviral expression vector comprises a CMV promoter that facilitates expression of the operably linked nucleotide sequence encoding C, prM and E in the packaging cell. However, it will be appreciated that other promoters may be useful in this regard, such as RSV, SV40, alpha crystallin, adenoviral and human elongation factor promoters, although without limitation thereto.

Accordingly a “flaviviral expression construct” is an expression vector into which a heterologous nucleic acid has been inserted so as to be expressible in the form of RNA and/or as an encoded protein.

Said heterologous nucleic acid may encode one or more peptides or polypeptides, or encode a nucleotide sequence substantially identical or substantially complementary to a target sequence.

The heterologous nucleic acid may encode any protein that is expressible in an animal cell.

With this in mind, the flaviviral replicon may be modified, adapted or otherwise engineered to be capable of including said heterologous nucleic acid, typically by the introduction of one or more cloning sites, as for example described in International Publication WO 99/28487.

Introduction of a tetracycline transactivator construct, packaging construct or flavivirus expression construct into an animal host cell may be by any method applicable to animal cells. Such methods include calcium phosphate precipitation, electroporation, delivery by lipofectamine, lipofectin and other lipophilic agents, calcium phosphate precipitation, DEAE-Dextran transfection, microparticle bombardment, microinjection and protoplast fusion.

It will be appreciated from the foregoing that the packaging system of the invention may be used for the expression of proteins in animal cells, preferably mammalian cells.

This may facilitate expression of any eukaryotic protein that requires post-translational processing and/or modification provided by animal cells. Non-limiting examples of such proteins include hormones, growth factors, transcription factors, enzymes, recombinant immunoglogulins or fragments thereof, antigens, immunogens and the like.

In a particular embodiment, VLPs produced according to the present invention may be used to infect appropriate animal cells and thereby facilitate expression of the encoded protein in the cells. Appropriate protein purification techniques may then be used to isolate and purify the expressed protein.

Such a system may exploit animal cells which are capable of expressing high levels of replicon-encoded heterologous protein, such as CHO cells although without limitation thereto.

In one particular embodiment, the heterologous nucleic acid may encode an immunogenic protein or peptide derived or obtained from pathogenic organisms such as viruses, fungi, bacteria, protozoa, invertebrates such as parasitic worms and arthropods or alternatively, may encode mutated, oncogenic or tumour proteins such as tumour antigens, derived or obtained from animals inclusive of animals and humans. Heterologous nucleic acids may also encode synthetic or artificial proteins such as immunogenic epitopes constructed to induce immunity.

Immunotherapeutic compositions of the invention may be used to prophylactically or therapeutically immunize animals such as humans.

Immune responses may be elicited or induced against viruses, tumours, bacteria, protozoa and other invertebrate parasites by expressing appropriately immunogenic proteins or peptide epitopes encoded by VLPs of the invention

Preferably, the immune response involves induction of CTL.

According to this embodiment, VLPs produced according to the invention may be used in the preparation of an immunotherapeutic composition or vaccine composition that further comprises an acceptable carrier, diluent or excipient and/or adjuvant.

By “pharmaceutically-acceptable carrier, diluent or excipient” is meant a solid or liquid filler, diluent or encapsulating substance that may be safely used in systemic administration. Depending upon the particular route of administration, a variety of carriers, well known in the art may be used. These carriers may be selected from a group including sugars, starches, cellulose and its derivatives, malt, gelatine, talc, calcium sulfate, vegetable oils, synthetic oils, polyols, alginic acid, phosphate buffered solutions, emulsifiers, isotonic saline and salts such as mineral acid salts including hydrochlorides, bromides and sulfates, organic acids such as acetates, propionates and malonates and pyrogen-free water.

A useful reference describing pharmaceutically acceptable carriers, diluents and excipients is Remington's Pharmaceutical Sciences (Mack Publishing Co. N.J. USA, 1991) which is incorporated herein by reference.

Any safe route of administration may be employed for providing a patient with the composition of the invention. For example, oral, rectal, parenteral, sublingual, buccal, intravenous, intra-articular, intra-muscular, intra-dermal, subcutaneous, inhalational, intraocular, intraperitoneal, intracerebroventricular, transdermal and the like may be employed.

As will be understood in the art, an “adjuvant” means one or more substances that enhances the immunogenicity and/or efficacy of a vaccine composition. Non-limiting examples of suitable adjuvants include squalane and squalene (or other oils of animal origin); block copolymers; detergents such as Tween®-80; Quil® A, mineral oils such as Drakeol or Marcol, vegetable oils such as peanut oil; Corynebacterium-derived adjuvants such as Corynebacterium parvum; Propionibacterium-derived adjuvants such as Propionibacterium acne; Mycobacterium bovis (Bacille Calmette and Guerin or BCG); interleukins such as interleukin 2 and interleukin 12; monokines such as interleukin 1; tumour necrosis factor; interferons such as gamma interferon; combinations such as saponin-aluminium hydroxide or Quil-A aluminium hydroxide; liposomes; ISCOM® and ISCOMATRIX® adjuvant; mycobacterial cell wall extract; synthetic glycopeptides such as muramyl dipeptides or other derivatives; Avridine; Lipid A derivatives; dextran sulfate; DEAE-Dextran or with aluminium phosphate; carboxypolymethylene such as Carbopol′ EMA; acrylic copolymer emulsions such as Neocryl A640 (e.g. U.S. Pat. No. 5,047,238); vaccinia or animal poxvirus proteins; sub-viral particle adjuvants such as cholera toxin, or mixtures thereof.

Pharmaceutical compositions inclusive of immunotherapeutic compositions and methods of immunization according to the invention may be administered to any animal inclusive of mammals and humans, although without limitation thereto.

Thus, veterinary and medical treatments are contemplated, which treatments may be administered therapeutically and/or prophylactically depending on the disease or ailment to be treated.

So that the invention may be readily understood and put into practical effect, reference is made to the following non-limiting examples.

### EXAMPLES

### Materials and Methods

Plasmids. The plasmid pEF-tTA-IRESpuro, a derivative of pEFIRES-P (Hobbs et al., 1998 Biochem Biophys Res Commun 252, 368-72) and containing sequence coding for the tetracycline transactivator (FIG. 1A) was a gift from Rick Sturm, University of Queensland). The plasmid pTRE2 CprME-IRESNeo (FIG. 1A) encoding KUN CprME gene cassette under the control of tatracycline-inducible promoter was constructed as follows. The sequence for the EMCV internal ribosome entry site (IRES) and the neomycin gene were excised from pBS-CIN4IN, a derivative of pCIN1 (Rees et al., 1996, BioTechniques 20 102-110) using MluI and XbaI. The IRESNeo cassette was then inserted into the corresponding MluI/XbaI sites of pTRE2 vector (Clontech) to produce an intermediate pTRE2IRESNeo plasmid. The sequence coding for the Kunjin (KUN) CprME gene cassette was PCR amplified by high fidelity Pfu DNA polymerase (Promega) from FLSDX plasmid DNA template {Khromykh et al., 1998, J. Virol. 72 5967) using the primers CprMEFor 5′ATTTAGGTGACACTATAGAGTAGTTCGCCTGTGTGA 3′ and CprMERev 5′GAGGAGATCTAAGCATGCACGTTCACGGAGAGA 3′ to produce a fragment with a BglII restriction enzyme site at the 5′ and 3′ end. It should be noted that the BglII site at the 5′ end of the fragment is located 100 nucleotides downstream of the forward primer and just upstream of the native KUN translation initiation codon. The BglII-BglII fragment containing KUN CprME sequence was then inserted into the BamHI site of pTRE2IRESNeo vector located upstream of the IRESNeo sequence to produce the pTRE2 CprME-IRESNeo plasmid (FIG. 1A).

The RNA-based KUN replicon vectors and other KUN replicon constructs encoding different heterologous genes that were used for in vitro transcription of different replicon RNAs have been previously (Khromykh & Westaway, 1997, J. Virol. 71 1497; Anraku et al., 2002, J. Virol. 76 3791; Liu, 2002 #1264; Varnavski & Khromykh, 1999, Virology 255 366; Varnavski et al., 2000, J. Virol. 74 4394). KUN replicon encoding M2 gene of respiratory syncytial virus (RSV) was constructed by cloning into RNAleu vector (Anraku et al., 2002, supra) of a DNA fragment containing RSV M2 cDNA sequence that was prepared by reverse transcription(RT) and PCR amplification of RNA from RSV-infected cells using appropriate primers.

The dengue virus type 2 (DEN2) replicon constructs pDENΔCprME and pDENΔprME were derived from the plasmid pDVWS601, which contains a full length cDNA clone corresponding to the genome of the New Guinea C strain of DEN-2 by creating large in frame deletions in the structural genes. pDENΔCprME retained the first 81 nucleotides of the C gene and the last 72 nucleotides of the E gene whilst pDENΔprME retained the first 21 nucleotides of the prM gene and last 72 nts of the E gene.

Cell lines, virus and antibodies. The BHK21 and Vero cell lines were cultured in Dulbecco's modified Eagle's medium (Life Technologies) supplemented with 10% fetal calf serum and penicillin/streptomycin at 37° C. with 5% CO₂. Wild type (wt) KUN virus, strain MRM61C, was grown in Vero cells as described previously (Westaway et al., 1997, J. Virol. 71 6650). Anti-KUN NS3 polyclonal antibodies raised in rabbits were described previously (Westaway et al., 1997, supra). The anti-KUN Envelope 3.91D monoclonal antibody (MAb) was raised in mice (Adams et al., 1995, Virology 206 49).

DNA transfection. BHK21 cells were cultured for 24 h in a 60 mm dish prior to transfection with 2 μg of plasmid DNA using Lipofectamine Plus reagent (Life Technologies) as described by the manufacturer.

Production of virus-like particles (VLPs) and determination of their titre. KUN replicon RNAs were transcribed in vitro using SP6 RNA polymerase and electroporated into tetKUNCprME cells essentially as described previously (Khromykh & Westaway, 1997, supra). Routinely, ˜30 μg of RNA were electroporated into 3×10⁶ cells. The electroporated cells were then seeded into a 100 mm dish and incubated in different volumes of medium at 37° C. for up to 8 days. Culture fluid (CF) was usually collected at 3-5 time points during this period and replaced with the same volume of fresh medium to allow multiple harvesting of VLPs. The titre of infectious VLPs was determined by infection of Vero cells with 10-fold serial dilutions of the collected CFs and counting the number of cells positive for NS3 expression in IF analysis with anti-NS3 antibodies performed at 30 to 40 h post-infection.

Immunofluorescence. Coverslips of cultured cells were fixed in 4% paraformaldehyde at 28-48 hr post-transfection with replicon RNAs or post-infection with VLPs and assayed for expression of KUN NS3 or E protein by indirect immunofluorescence (IF) with anti-NS3 or anti-E antibodies, respectively.

Northern blot analysis. Total RNA was extracted from tetKUNCprME cells cultured with and without doxycycline and from normal BHK21 cells using Trizol reagent (Life Technologies). 20 μg of RNA was separated on a 1% formamide-TAE agarose gel and then transferred to Hybond-N (Amersham-Pharmacia Biotech) by capillary blotting using 20×SSC. An AflII-PstI fragment isolated from pTRE2INeoCprME was used as the template for preparation of labelled probe. This ³²P-labelled probe was prepared using the Rediprime II kit (Amersham-Pharmacia Biotech) as described by the manufacturer. The RNA was hybridised with the ³²P-labelled DNA probe using ExpressHyb solution (Clontech) at 68° C. essentially as described by the manufacturer. Bands were visualised by exposure to X-ray film or by phosphorimaging, and quantitated using the ImageQuant software (Molecular Dynamics).

Western blot analysis. tetKUNCprME cells were cultured for 2 days in a 60 mm dish with and without doxycycline and cellular proteins were extracted using Trizol reagent as described by the manufacturer. BHK21 cell proteins were also recovered for use as a negative control. The protein concentration for each sample was determined using the BioRad Protein assay (BioRad) as described by the manufacturer. Five μg of total cell protein was separated on a 12.5% gel by SDS-PAGE and transferred onto Hybond-P membrane (Amersham-Pharmacia Biotech, UK). The membrane was incubated overnight at 4° C. in blocking buffer (5% skim milk/0.1% Tween 20 in phosphate-buffered saline (PBS)). The KUN anti-E MAb was diluted 1:10 in blocking buffer and incubated with the membrane for 2 h at room temperature. The membrane was washed 3 times with 0.1% Tween-20/PBS for 5 min, then the secondary antibody was added. The secondary antibody, goat anti-mouse horseradish peroxidase, was diluted 1:2000 in blocking buffer and incubated with the membrane for 2 h at RT. The membrane was again washed with 0.1% Tween-20/PBS and developed using the ECL +Plus kit (Amersham-Pharmacia Biotech). The membrane was then exposed to X-ray film for varying time intervals.

RT-PCR and sequencing. Total RNA was extracted from a 60 mm dish of tetKUNCprME cells using Trizol. 0.1 μg of RNA was reverse-transcribed and amplified using a One-Step RT-PCR kit (Invitrogen). The oligonucleotide primers used were to the KUN cprME region with the forward primer, CoreXbaI 5′GGCTCTAGACCATGTCTAAGAAACCAGGA3′ and the reverse primer, cprMERev 5′GAGGAGATCTAAGCATGCCGTTCACGGAGAGA3′. The cDNA product was then used as a template for sequencing with BigDye Terminator Mix (Applied Biosystem) using 6 different primers to cover the full sequence of this region.

KUN VLP and IL-2 combinational tumour therapy. For groups of female C57BL/6J mice (6-8 weeks old, n=3 per group) were injected with 5×10⁴ LLOva tumour cells (Nelson et al., J Immunol. 2001, 166 5557-66) s.c. on the back, four tumours per mouse (n=12 tumours per group). Once the tumours became palpable (>1×1 mm²), 2 groups of mice were injected with 10⁸ pfu (in 200 □1) KUN VLPMpt and the other 2 Control groups were injected with PBS, both by the i.p. route 2 times separated by 10 days. One group from VLPMpt and Control received 2 doses of 2000 IU of murine IL-2 by the i.p. route separated by 2 days, 4 days after the first VLPMpt or PBS injection. The other 2 groups did not receive IL-2. The tumour size was recorded every day and the mice were euthanised when the tumour size reached 15×15 mm² (Anraku et al., 2002, supra).

Assessing an ability of amplification and spread of KUN replicon-virus like particles (VLPs) in KUN tetKUNCprME replicon packaging cell line (A8 cell line).

Cells: Normal BHK21 cells and KUN tetKUNcPrME KUN replicon packaging cells (A8 cell line) (Harvey et al, J Virol. 2004 78 531-8), incorporated herein by reference, were grown in Dulbecco minimal essential medium (DMEM; Invitrogen, San Diego, Calif.) supplemented with 10% fetal bovine serum (FBS) at 37° C. in a CO₂ incubator.

KUN replicon VLPs: The preparation of KUN repPAC/β-gal replicon VLPs were described in (Harvey et al, J Virol. 2004, supra). Briefly, A8 cells were electroporated with in vitro transcribed KUN repPAC/β-gal RNA, which encode a β-galactosidase gene for easy comparison of gene expression and a puromycin resistance gene for selection. The cell culture fluid were collected at different time point after RNA transfection and the titer of the VLPs comprising encapsidated replicon KUN repPAC/β-gal RNA in the harvest fluid were calculated by the β-gal positive cell number by infecting Vero cells and staining them with X-Gal 48 hours after infection

KUN repPAC/β-gal replicon VLPs infection, X-Gal staining and β-gal assay. BHK21 and KUN KUN repilcon packaging A8 cells in 24-wells plate at 90% confluent were infected with repPAC/β-gal VLPs at a multiplicity of infection (MOI) 1 and incubated in the medium without doxcyline. 48, 96 and 144 hours after infection, cells were fixed by 4% formaldehyde-phosphate-buffered saline and were stained in situ with 5-bromo-4-chloro-3-indolyl-β-D-galactopyopyranoside (X-Gal) or cells were trypsined, counted and lysed for a 13-Gal assay by using a commercial β-gal detection kit according to the instruction described by the manufacturer (Promega, Madison Wis.).

### RESULTS

Establishment of the tetracycline-inducible BHK cell line, tetKUNCprME, capable of packaging KUN replicon RNA into VLPs. To our knowledge, no stable cell lines simultaneously expressing all three flavivirus structural proteins have been reported to date. We have previously generated a Vero cell line stably expressing KUN C protein, however, the level of expression was low (Westaway et al., 1997. Virology. 234 31-41). Our previous attempts to generate a stable cell line continuously expressing all three KUN structural genes under control of separate promoters (expressing C and prM-E separately), using standard (non-inducible) DNA expression vectors, resulted in great instability of expression, producing only 10-20% positively expressing cells after a few cell passages (not shown). Attempts to use these cell lines to produce KUN replicon VLPs resulted in very low VLP titres (data not shown).

Initially, BHK21 cells were transfected with pEF-tTA-IRESpuro plasmid DNA, a derivative of pEFIRES-P (Hobbs et al., 1998, Biochem Biophys Res Commun. 252368-372) containing a sequence coding for the tetracycline transactivator (FIG. 1A), to establish a BHK cell line, BHK-Tet-Off, stably expressing the tetracycline transactivator. Two days following transfection the antibiotic puromycin at a concentration of 10 μg/ml was added for selection of cell clones. Five cell clones were isolated and cultured successfully from this transfection. These clones were then analysed for induction of expression by transfection with the plasmid, pTRE2luciferase (Clontech) in the presence (0.5 μg/ml) or absence of doxycycline (an antibiotic of the same spectrum as tetracycline but with higher specific activity and longer half life). All BHK-Tet-off cell clones demonstrated varying degrees of induction and background levels (results not shown). Two BHK-Tet-Off cell clones displaying the highest fold induction of luciferase expression compared to uninduced cells were used to establish a stable BHK cell line expressing the KUN structural proteins, core (C), membrane (prM) and envelope (E). The cells were transfected with pTRE2CprME-IRESNeo plasmid DNA (FIG. 1A) constructed by subcloning KUN CprME gene cassette and the encephalomyocarditis virus internal ribosomal entry site—neomycin phosphotransferase gene cassette (IRESNeo) into the pTRE2 vector (Clontech, North Ryde, Australia). Transfected cells were subjected to selection with 0.5 mg/ml of Geneticin (G418) in media that also contained 10 μg/ml puromycin and 0.5 μg/ml of doxycycline to establish stable packaging cell lines. To select the most efficient packaging cell line, a number of cell clones conferring resistance to G418 and puromycin were electroporated with KUN replicon RNA (RNAleu) and cultured without doxycycline to determine whether they were able to produce infectious KUN replicon VLPs. The titres of infectious VLPs (in infectious units (IU) per ml) present in harvested culture fluids (CFs) were determined by infection of Vero cells followed by immunofluorescence analysis with anti-NS3 antibodies as described previously (Khromykh et al., 1998, J Virol. 72 7270-7279; Westaway et al., 1997, J Virol. 71 6650-6661)

Four cell clones, i.e. #A3, #A8, #E1 and #E5, were capable of VLP production with efficiencies varying from 5×10⁴ to 2×10⁸ IU per ml at 53 h after RNA electroporation (Table 1). The most efficient cell clone #A8 producing 2×10⁸ IU/ml of VLPs was designated tetKUNCprME and used in all further studies. The identity of the KUN CprME sequence encoded in the mRNA produced in tetKUNCprME cells to that of the wild type KUN CprME sequence was confirmed by sequencing the entire CprME region after reverse transcription (RT)-PCR amplification of total RNA isolated from tetKUNCprME cells. No nucleotide changes from the sequence present in the plasmid DNA pTRE2 CprME-IRESNeo were found.

CprME expression and optimization of production of secreted KUN replicon VLPs in tetKUNCprME cells. To examine levels of secreted KUN proteins and KUN VLPs in the culture fluid of tetKUNCprME cells we used an antigen capture ELISA as previously described (Hunt et al., 2002, supra). CFs collected from induced and uninduced tetKUNCprME cells that were cultured for 48 h prior to analysis showed no detectable levels of KUN E protein in both CF samples (FIG. 1B). However, when the cells were electroporated with RNAleu replicon RNA, a dramatic increase in ELISA readings was noticed by 45 h after RNA electroporation in the CF sample from induced cells, while only a marginal increase in ELISA readings was detected in the CF sample from uninduced cells (FIG. 1B). When VLPs in these CF samples were titrated on Vero cells, the titres of VLPs correlated well with the ELISA results. 500 IU of VLPs per ml detected in the CF samples collected from uninduced cells produced an ELISA reading OD₄₅₀ of ˜0.11, while 2.1×10⁸ IU of VLPs per ml in the CF sample from induced cells gave an ELISA reading of ˜0.63 (FIG. 1B).

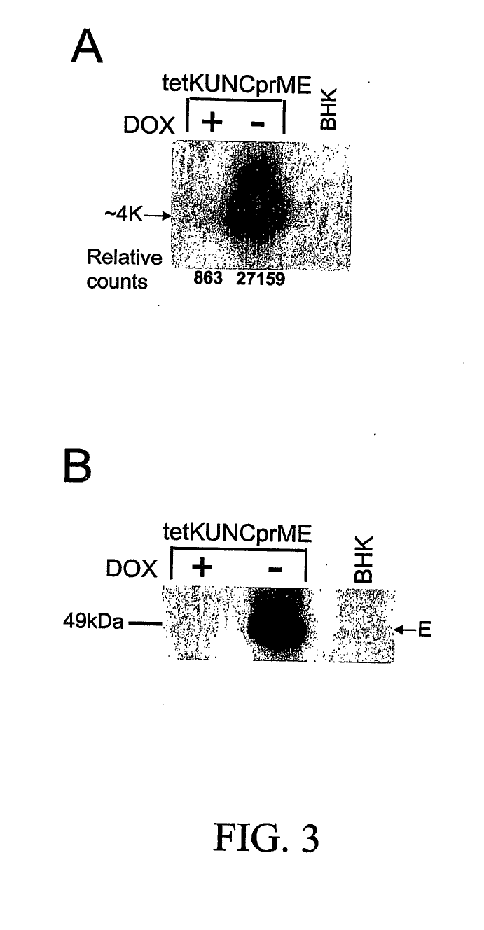

In order to examine the levels of CprME mRNA transcription and intracellular expression of the CprME genes in the tetKUNCprME cell line, cells were cultured for 48 h with and without doxycycline in the media. Normal BHK21 cells were included as a negative control. The CprME mRNA transcription was analysed by Northern blot hybridisation of total cell RNA with a ³²P-labelled CprME-specific cDNA probe (FIG. 3A) and the expression of KUN proteins was analysed by Western blot analysis with KUN anti-E antibodies (FIG. 3B). The results showed that there was very little of CprME mRNA and KUN E protein produced in the presence of doxycycline (uninduced cells). In contrast, removal of doxycycline resulted in 30 fold increase in the level of CprME mRNA, as judged by the relative phosphoimager counts in the corresponding labelled bands (FIG. 3A). Approximately similar increase in the level of KUN B protein production was also detected (FIG. 3B).

In order to optimize VLP production, studies were performed with the harvesting of culture fluid and the removal of doxycycline from the media at different time points. Following electroporation of KUN replicon RNA (RNAleu), media containing doxycycline (0.5 ug/ml) was added to the cells for a further 16 h or 30 h and then replaced with fresh media without doxycycline. A 60 mm² dish of electroporated cells was set up continually without doxycycline for comparison. The culture fluid was harvested from each dish at 53 h and 68 h post-electroporation and examined by infectivity assay on Vero cells. The results showed that the optimal time for removal of doxycycline to induce CprME expression for VLP production was immediately after RNA electroporation (Table 2). A delay in the removal of doxycycline from the media resulted in a substantial decrease in the amount of VLPs produced.

To determine the optimal VLP harvesting protocol and the ability of tetKUNcprME cells to produce high levels of VLPs encoding various heterologous genes, KUN replicon RNA RNAleu and replicon RNAs encoding different heterologous genes such as murine polytope (RNAleuMpt), HIV-1 gag (KUNgag), puromycin acetyl transferase (repPAC), puromycin acetyl transferase and β-galactosidase (repPACP-gal), and green fluorescence protein (repGFP) (Anraku et al., 2002, supra; Liu et al., 2002, J Virol. 76 10766-10775; Varnavski & Khromykh, 1999, Virology 255 366-375; Varnavski et al., 2000, 3 Virol. 74 4394-4403) were electroporated into tetKUNCprME cells. VLPs were harvested at different times after RNA electroporation and the medium was replaced with fresh medium every time VLPs were harvested to allow multiple harvesting of VLPs (Table 3). Nearly all the VLP titres from day 3 onwards after electroporation were in the range of 10⁷ to 10⁹ IU per ml, and remained high even in the third or fourth consecutive harvests up to 10 days after transfection, depending on the nature of the replicon RNA and the VLP harvesting protocol (Table 3). The total production of VLPs from the initially transfected 3×10⁶ tetKUNCprME cells using the most optimal VLP harvesting protocol reached 5.4×10¹⁰ infectious particles (repPACβ-gal RNA exp 2 in Table 3) and was in the range from 1.6×10⁹ to 1.3×10¹⁰ infectious particles per 3×10⁶ electroporated cells when other harvesting protocols and different KUN replicon RNAs were used (Table 3).

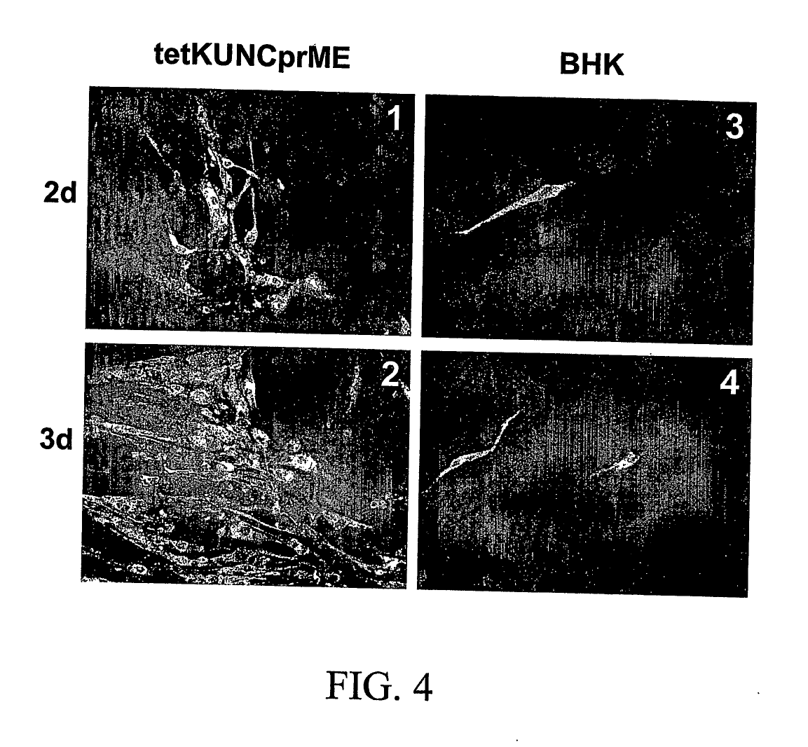

To examine whether KUN replicon VLPs can be amplified by spread in tetKUNCprME cells but not in normal BHK cells the cells were infected with RNAleuMpt VLPs at low MOI (0.1) and incubated in the medium without doxycycline. IF analysis of infected cells with KUN anti-NS3 antibodies showed significant increase in the size of positive cell foci from day 2 to day 3 post-infection (FIG. 4, panels 1 and 2) demonstrating amplification and spread of VLPs in tetKUNCprME cells. In contrast, only individual positive cells were detected in infected normal BHK21 cells at both day 2 and day 3 after VLP infection (FIG. 4, panels 3 and 4). In a separate experiment, an approximately 10-fold increase in VLP titres from day 3 to day 5 of incubation after infection of tetKUNCprME cells with 0.1 MOI of RNAleuMpt VLPs was detected (results not shown), thus further confirming amplification of VLPs by spread in the packaging cells.

The results convincingly demonstrate that the tetKUNCprME cell line is able to produce substantially (˜1500-fold) higher amounts of KUN replicon VLPs compared to our previously published protocol using the cytopathic SFV replicon for expression of KUN structural genes (Varnavski & Khromykh, 1999, supra).

This should be compared to a report on the generation of a CHO cell line stably expressing tick-borne encephalitis (TBE) prME genes and its use for packaging of TBE replicon RNA having deleted prME genes (Gehrke et al, 2003, J. Virol. 77 8924-8933). The highest titres of secreted TBE replicon VLPs obtained in prME-expressing CHO cells were 5×10⁷ IU/ml. That is 32-fold lower then the highest titres of KUN replicon VLPs obtained in tetKUNCprME cells (1.6×10⁹ IU/ml, Table 3). Moreover, the total maximum amount of TBE replicon VLPs produced per 10⁶ transfected cells was ˜10⁸ IU, which is ˜540-fold less than that obtained for KUN replicon VLPs (5.4×10¹⁰ IU, see Table 3). It is however, difficult to do any further comparison of the packaging efficiencies between these two systems in view of the differences in cell lines used (CHO for TBE and BHK for KUN), replicon RNAs (with core gene for TBE and without core gene for KUN), electroporation conditions (i.e. number of transfected cells, RNA quantities not reported for TBE RNA, and electroporator settings), and protocols for harvesting VLP.

However, it is clear that tetKUNCprME cells of the present invention, offer the flexibility of inducible expression, apparently higher titres, continuous harvesting, and higher total amounts of produced replicon VLPs. In addition, tetKUNCprME cells were capable of packaging replicon RNAs from different flaviviruses (see below).

Stable expression of KUN structural proteins in tetKUNCprME cells. To determine the stability of expression of the KUN CprME genes, tetKUNCprME cells were cultured for 12 passages without puromycin and G418 and then electroporated with KUN replicon RNA (RNAleu) to determine the efficiency of VLP production. Doxycycline was present in the medium during passaging to ensure suppression of CprME expression. tetKUNCprME cells that were cultured for 12 passages in the presence of all three antibiotics, i.e. puromycin, G418 and doxycycline, were electroporated in parallel to compare VLP production efficiency. Doxycycline was removed from the medium immediately after electroporation of a replicon RNA to induce expression of CprME and enable VLP production. Titres of VLPs collected at 48 h after replicon RNA ransfection from cells that were maintained under puromycin and G418 selection during passaging were similar to the titre of VLPs collected at the same time from cells that were maintained without puromycin and G418 selection (2.2×10⁶ IU/ml and 1.7×10⁶ IU/ml, respectively). Although the VLP titres in this particular experiment were lower then in the majority of the other packaging experiments, the results clearly demonstrate the stability of expression of KUN structural proteins in tetKUNCprME cells after at least 12 passages in the absence of antibiotic selection and thus indicate stable integration of KUN structural gene cassette into the cell genome.

Absence of infectious KUN virus in replicon VLP preparations. The presence of overlapping sequences in the C-terminal region of C gene and the N-terminal region of E gene of KUN replicon RNA and of CprME mRNA produced in tetKUNCprME cells may potentially promote homologous recombination that may lead to production of infectious KUN virus in VLP preparations. In a previously developed packaging system we eliminated any possibility of potential recombination by separating expression of C gene and prM-E genes from two different mRNAs produced from SFV replicon vector. However, our numerous complementation experiments with KUN RNAs (for a summary see Khromykh, 2000, supra} as well as complementation experiments with YF RNAs (Lindenbach & Rice, 1997, J Virol. 71 9608-17; Lindenbach & Rice, 1999, J Virol. 73 4611-21) where extended regions of complementarity were present between defective and helper RNAs failed to detect any recombinant infectious viruses that could have been generated by homologous recombination. To examine whether any recombined replication competent KUN virus was produced during production of KUN replicon VLPs in tetKUNCprME cells, CFs harvested at 2 days after transfection with RNAleu RNA were used to infect Vero cells grown on coverslips. The infected cells were incubated for 5 days and examined for expression of E protein by immunofluorescence. The tissue culture fluid from the infected coverslips was then passaged again on fresh cultures of Vero cells for a further 5 days and examined by IF with anti-E antibodies. No E-positive cells were detected in both passages (results not shown). Parallel labelling with anti-NS3 antibodies showed numerous positive cells in the first passage, but no positive cells in the second passage (results not shown) demonstrating that VLPs deliver replicon RNA only in the first round of infection. Similarly, packaging of TBE replicon RNA in CHO cells stably expressing prM-E genes did not result in production of any infectious TBE virus even after several passages in the packaging cell line, despite the overlap in viral genomic sequences between prM-E and replicon RNAs.

Additional evidence of the absence of infectious KUN virus in VLP preparations was sought by the most sensitive method for virus detection, intracranial injection of suckling mice. Groups of ten 2-3 day old Balb/C suckling mice were inoculated intracranially with 4×10⁶ IU of KUN-MPt VLPs or with 1 pfu of wt KUN virus (strain MRM61C) as a positive control. All ten mice injected with 1 pfu of wt KUN virus developed paralysis of the hindlegs at 4 days post inoculation and had to be sacrificed. In contrast, all VLP-injected mice remained healthy and demonstrated normal development for the duration of the experiment (21 days). These in vitro and in vivo results with KUN replicon VLPs and the in vitro results with TBE replicon VLPs (Gehrke et al., 2003, supra) clearly demonstrate that production of flavivirus replicon VLPs in packaging cells expressing continuous structural gene cassettes does not lead to the generation of any recombinant infectious virus. In comparison, a 10⁸ IU of Sindbis virus replicon VLPs produced in BHK packaging cell line expressing a continuous Sindbis virus structural gene cassette, contained ˜10⁵ pfu of infectious viruses generated by recombination (Polo et al., 1999, Proc Natl Acad Sci USA. 96 4598-4603). Splitting the structural genes into two separate expression cassettes in the packaging cell line appeared to remove contamination with infectious viruses to an undetectable level, but at the same time reduced the titres of replicon VLPs to 5×10⁶-1×10⁷ VLPs per ml (Polo et al., 1999, supra)

Packaging of West Nile and dengue virus replicons into secreted infectious VLPs in tetKUNCprME cells. To examine whether tetKUNCprME cells can be used to package replicon RNAs derived from other flaviviruses, we used replicon RNAs from a closely related West Nile (WN) virus and from a distantly related dengue type 2 (DEN2) virus. The WN replicon construct “Replicon” with a large deletion in structural region, retaining only the first 20 codons of C gene and the last codons of E gene, was described previously (Shi et al., 2002, Virology. 296 219-233; Lo et al., 2003, J Virol. 77 10004-10014).

The dengue virus type 2 (DEN2) replicon constructs pDENΔCprME and pDENΔprME were derived from the plasmid pDVWS601, which contains a full length cDNA clone corresponding to the genome of the New Guinea C strain of DEN-2 (Pryor et al., 2001, Am J Trop Med Hyg. 65 427-434) by creating large in frame deletions in the structural genes. pDENΔCprME retained the first 27 codons of the C gene and the last 24 codons of the E gene whilst pDENΔprME retained the entire C gene, the first 7 codons of the prM gene and the last 24 codons of the E gene.

For packaging experiments, DENΔCME or DENΔME replicon RNAs were electroporated into teKUNCprME cells and incubated in the medium without doxycycline. KUN replicon RNA (RNAleu) was included for comparison of VLP production. IF analysis with cross-reacting KUN anti-NS3 antibodies at 2d after transfection showed ˜80% and 95% of positive cells after transfection with DENΔME and DENΔCME RNAs, respectively. Transfection of KUN replicon RNA RNAleu resulted in ˜95% of NS3-positive cells. Culture fluid was collected at 2d post-electroporation and titrated by infectivity assay on Vero cells. The titre of infectious VLPs produced from DENΔME and DENΔCprME replicon RNAs were 8×10⁴ IU/ml and 1.8×10⁵ IU/ml respectively. The KUN replicon RNA in the same experiment produced VLPs with a titre of 2.2×10⁷ IU/ml.

In a separate experiment, electroporation of WN replicon RNA into tetKUNCprME cells resulted in detection of ˜70-80% of NS3-positive cells and production of 7×10⁷ IU/ml of secreted VLPs by 4d post-electroporation. Electroporation of KUN replicon RNA RNAleu performed in the same experiment resulted in detection of ˜80-90% of NS3-positive cells and production of 10⁸ IU/ml of VLPs by day 4 post-electroporation.

The successful generation of chimeric flaviviruses by replacing structural genes from one virus with those from other flaviviruses demonstrates that structural proteins from one flavivirus are capable of packaging RNA of another flavivirus when they are expressed in cis from the same RNA molecule. Our results, represent the first demonstration of packaging of different flavivirus replicon RNAs by the KUN structural proteins provided in trans. Given very high homology between KUN and NY99 strain of WN virus (Lanciotti et al., 1999, Science. 286 2333-2337; Liu et al., 2003, supra) and their relatively similar replication efficiencies, the observed similar packaging efficiencies of KUN and WN replicon RNAs are not surprising. The ˜100-fold lower packaging efficiency of DEN2 replicon RNAs compared to that of KUN replicon RNA could be attributed to a number of factors, including significant sequence differences between these two viruses, and lower replication efficiencies of dengue viruses in general. Previous experiments with full-length infectious DEN2 cDNA showed relatively inefficient production of secreted DEN2 virus directly after RNA transfection into BHK cells Gualano et al., 1998, J Gen Virol. 79 437-446). Although we did not compare the efficiencies of replication of DEN2 and KUN replicon RNAs in tetKUNCprME cells, it is likely that replication of DEN2 replicon RNAs would be less efficient than KUN replicon RNA leaving less RNA available for packaging. Optimal packaging may also require specific interactions between RNA and core protein of the same virus, however, no signals/motifs in flavivirus RNA or core protein that determine specificity of packaging have yet been defined. The current packaging system is likely to contribute to future studies of packaging signals and increase understanding of how flavivirus virions are assembled and secreted.

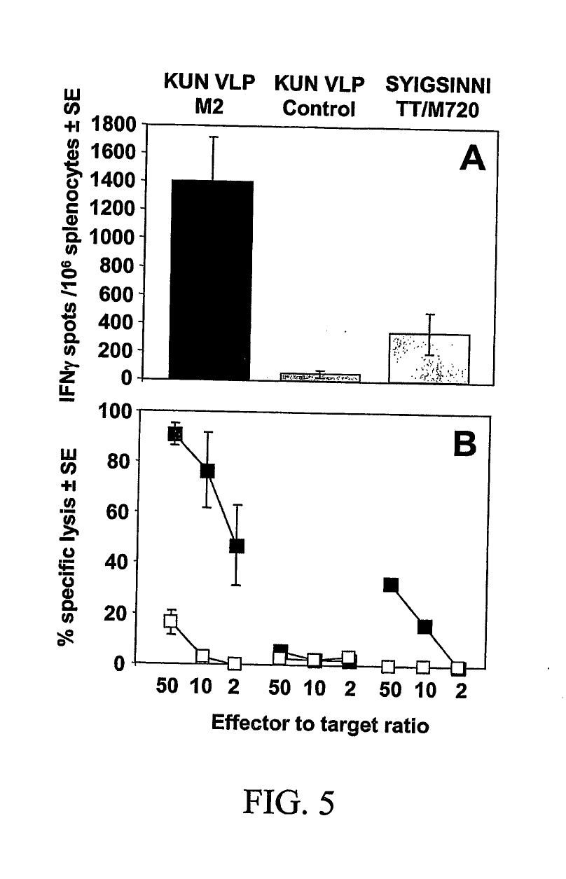

Immunization with high doses of KUN replicon VLPs prepared in tetKUNCprME cells improves CD8+ T cell responses to encoded immunogens. The packaging cell line allowed production of KUN replicon VLPs with ˜100-fold higher titres, thus enabling testing of increasing doses of VLPs in immunization experiments. A ten-fold increase in the dose of KUN replicon VLPs encoding murine polytope (KUN-Mpt VLPs) from 10⁶ to 10⁷ IU of VLPs, induced 3 to 4 fold more SIINFEKL epitope-specific CD8 T cells as measured by ex-vivo IFNγ ELISPOT assay (FIG. 5A). A further ten-fold increase from 10⁷ to 10⁸ IU of VLPs resulted only in a marginal increase in the number of SIINFEKL-specific CD8 T cells induced (FIG. 5A). In a separate experiment, BALB/c mice were immunized once with 2.5×10⁷ IU of KUN VLPs encoding the respiratory syncytial virus (RSV) M2 gene. KUN replicon encoding the RSV M2 gene was constructed by cloning into the RNAleu vector a DNA fragment containing RSV M2 cDNA sequence that was prepared by reverse transcription (RT) and PCR amplification of RNA isolated from cells infected with RSV A2 isolate. Highly potent CD8+ T cell responses specific for the RSV M2 epitope, SYIGSINNI, were generated, with ELISPOT analysis showing an average of 1400 spots per 10⁶ splenocytes (FIG. 5B, KUN-M2 VLP), and a standard chromium release showing over 45% specific lysis after effectors were diluted to an effector:target ratio of 2:1 (FIG. 5C, KUN-M2 VLP). These responses exceeded those reported following vaccination with a replication competent recombinant vaccinia virus encoding RSV M2 (Aung et al., 1999, J Virol. 73 8944-8949; Kulkarni et al., 1993, J Virol. 67 4086-4092; Simmons et al., 2001, J Immunol. 166 1106-1113)

A control KUN VLP failed to induce significant specific responses (FIGS. 5B and 5C, KUN VLP Control), and a peptide-vaccine formulated with SYIGSINNI-peptide induced several fold lower responses (FIGS. 5B and C, SYIGSINNI/TT/M720).

High titre KUN VLPs for therapeutic vaccine treatment in cancer. The cure of established tumours by CD8 T cell based therapy requires very large numbers of anti-cancer CD8 T cells (Overwijk et al., 2003, J Exp Med. 198 569-80). We have shown herein that more CD8 T cells are induced by increasing doses of VLPs. Thus we sought to determine whether high dose VLP vaccination could be used therapeutically to mediate significant anti-cancer activity. In this model VLPs encoding the murine polytope (Mpt) which contains the ovalbumin CD8 T cell epitope SIINFEKL, were used as a therapeutic against Lewis Lung cells expressing the model tumour antigen ovalbumin (LLOva; Nelson et al., 2001, supra).

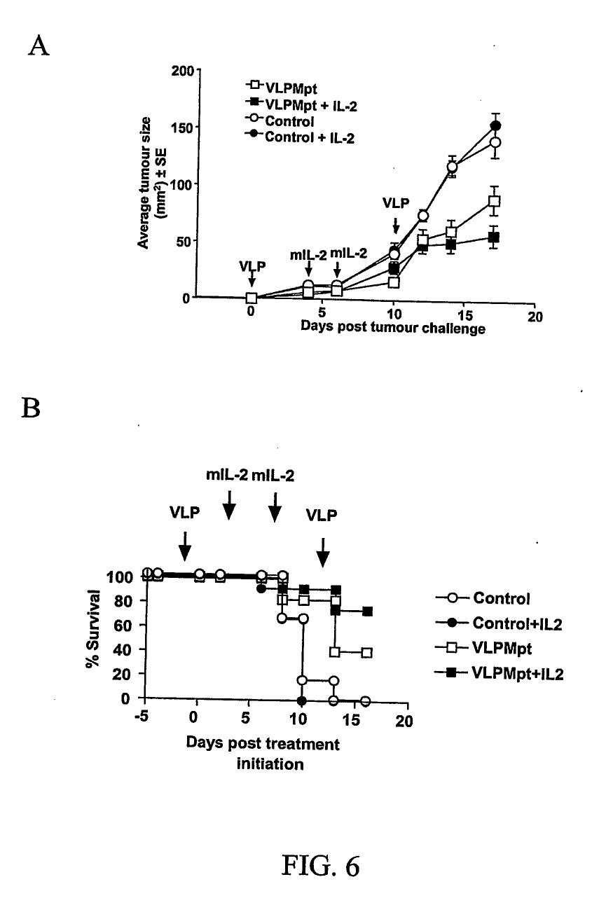

Groups of mice with established LLOva tumour were vaccinated ip with 10⁸ KUN VLPMpt (FIG. 6. VLPMpt) or PBS (FIG. 6. Control) twice, with and without IL-2 at the times indicated (FIG. 6, arrows). VLPMpt vaccination significantly slowed the growth of the tumours. IL-2 alone or in combination with the VLP vaccination did not significantly affect tumour growth.

Therefore, it is concluded that therapeutic administration of a high titre VLPMpt vaccine, which is capable of inducing high levels of SIINFEKL-specific CD8 T cells was able to slow significantly the growth of pre-existing LLOva tumours. IL-2 had no significant effect, either alone or in combination with VLP treatment.

Packaging of KUN replicon RNAs with adaptive mutations in NS2A and NS5 into virus-like particles. A previous report showed that Sindbis virus and SFV replicon RNAs with some of the adaptive (noncytopathic) mutations in nsP2 could not be packaged efficiently into VLPs while those RNAs with other adaptive mutations in nsP2 could (Perri et al., 2000, J Virol. 74 9802-9807).

We examined the packaging ability of KUN replicon RNAs with adaptive mutations by transfecting them into our recently reported tetracycline-inducible packaging BHK cell line tetKUNCprME. The secreted VLPs were harvested every 2 days for 6 to 8 days and the VLP titres were determined as described in Materials and Methods. Replicon RNA with the NS2A/A30P mutation was packaged with efficiency similar to that of the wt RNA; the other mutant RNAs suffered a 50- to 500-fold decrease in packaging efficiency at day 2, but all except that with the combined mutations in NS2A recovered packaging efficiency close to the wild type by day 6 (Table 4). The RNA with combined mutations in NS2A was still packaged 40-fold less efficiently than the wild type RNA by day 8. In summary, only the NS2A/A30P mutation did not affect packaging efficiency of replicon RNA, while other adaptive mutations decreased the packaging efficiency.

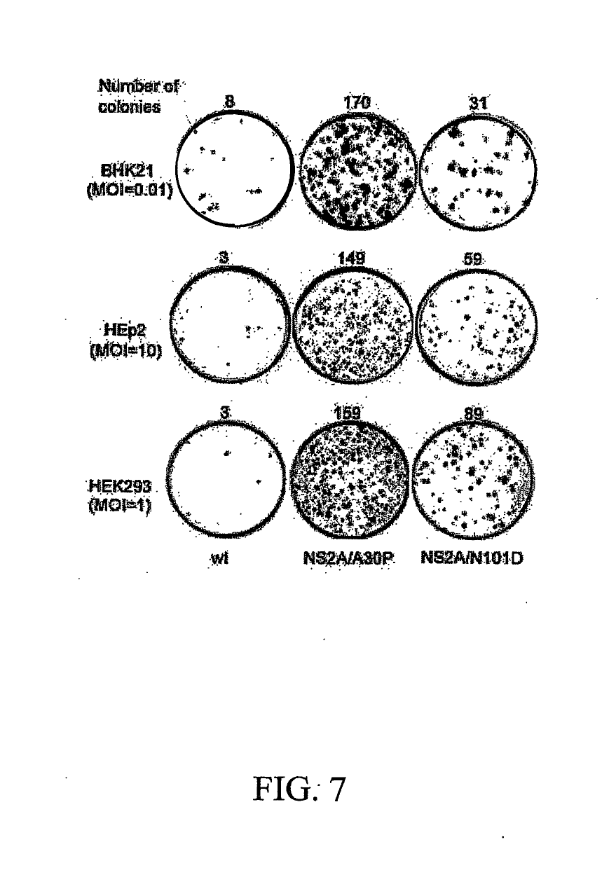

Use of VLPs obtained in tetKUNCprME cells to generate stably expressing cell lines. We next examined whether the adaptive mutations in NS2A shown to provide an advantage in establishing persistent replication in the hamster cell line, BHK21, would also provide a similar advantage in other cells lines, particularly human cell lines. Monolayers of two human cell lines, HEK293 and HEp-2 were infected with VLPs containing packaged wt and mutated replicon RNAs at MOI of 1 and 10, respectively (titrated on Vero cells), and propagated for 7 days in the medium with 1 μg/ml of puromycin. X-gal staining of puromycin-resistant colonies showed a ˜50-fold increase in the number of colonies relative to wild type replicon for the NS2A/A30P mutant and ˜20-fold increase for the NS2A/N101D mutant in both HEK293 and HEp-2 cells (FIG. 7). Similar differences in the number of puromycin-resistant colonies between the wt and NS2A-mutated replicon RNAs were observed in BHK cells after infection with 0.01 MOI of replicon VLPs (FIG. 7). Interestingly, infection of HEK293 and HEp-2 cells required 10- and 100-fold more VLPs, respectively, to produce similar numbers of puromycin resistant colonies to those produced in BHK21 cells (FIG. 5). Similar differences between these cell lines were observed in the efficiency of replication of wild type KUN virus (not shown). In separate experiments, ˜20-fold more efficient replication of wild type KUN virus was observed in Vero cells compared to that in BHK cells (results not shown). The results confirmed the advantage relative to the parental replicon RNAs with adaptive mutations in NS2A in their ability to establish persistent replication in different cell lines. Propagation of replicon VLP-infected BHK, Vero, HEK293 and HEp-2 cells in the selective medium with puromycin resulted in the establishment of cell populations stably expressing wt and NS2A-mutated replicon RNAs with retention of mutations confirmed by sequencing (not shown). In all the experiments with BHK, Vero, HEK293, and HEp-2 cells, NS2A/A30P mutation allowed more efficient and quicker establishment of stably expressing cell lines (result not shown). In agreement with the results in BHK cells, the efficiencies of RNA replication and β-gal expression in established puromycin-resistant cell lines in HEp-2 and 293 cells stably expressing different replicon RNAs were also similar (results not shown).

Use of tetKUNCprME packaging cells for enhanced expression of heterologous genes from Kunjin replicon vector.

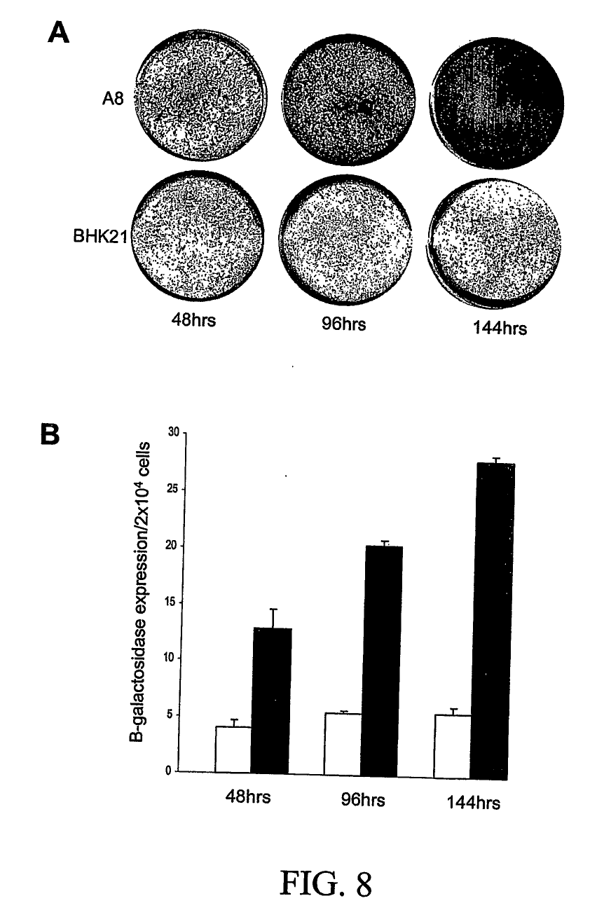

To examine whether KUN replicon VLPs can be amplified by spread in KUN packaging A8 cells but not in normal BHK cells, the cells were infected with repPAC/β-gal VLPs at multiplicity of infection (MOI) 1 and incubated in the medium without doxycycline. X-gal staining analysis of infected A8 packaging cells showed a significant increase in the number of β-gal positive cells from day 2 (48 hours) to day 6 (144 hours) postinfection (FIG. 8A), demonstrating amplification and spread of β-gal VLPs in A8 cells. In contrast, only individual positive cells were detected in infected normal BHK21 and the β-gal positive cell number were barely changed between day 2 and day 6 cells after KUN-replicon VLP infection (FIG. 8A). In addition, the β-gal positive cell numbers in KUN repPAC/β-gal VLPs infected A8 KUN packaging cells is much more than that in normal BHK21 cells from day 2 post infection (FIG. 8A), indicated the amplification and spread replicon VLPs in the early time of day 2.

β-gal analysis of lysed KUN replicon VLPs infected cells showed an approximately three-fold increase of β-gal expression from day 2 to day 6 infection of incubation after infection of A8 cells (FIG. 8B), in contrast only 1.3 fold increase of β-gal expression from day 2 to day 6 infection of incubation after infection of normal BHK21 cells. The rational of β-gal expression of repPAC/β-gal replicon VLPs infected A8 packaging cells: normal BHK21 cells from day 2 to day 6 were increased from 2.3 to 5.2 fold, thus further confirming amplification of VLPs by spread in the KUN replicon packaging cells. This relatively modest increase (3 to 5 fold) of β-gal expression observed from day 2 to day 6 of infection could be due to the impaired ability of newly infected aged (2 to 6 days old over-confluent) A8 packaging cells to support efficient KUN RNA replication, not necessarily represent inefficient spread of VLPs. The researchers have previously observed a lower efficiency of KUN RNA replication in aged BHK cells compared to that in actively dividing BHK cells in many experiments. In conclusion, the data show that the KUN RNA replicon VLPs can be amplified and spread in replicon packaging cells (A8 cell line).

### DISCUSSION

We have described here a novel packaging system for encapsidation of flavivirus replicon RNAs into virus-like particles using a tetracycline-inducible stable packaging cell line tetKUNCprME expressing KUN virus structural genes. High titres of VLPs reaching up to ˜4×10⁸ VLPs per ml, and multiple VLP harvests for up to 10 days allowed a total yield of up to ˜6.5×10⁹ VLPs from a single initial electroporation of 3×10⁶ cells. This represents a substantial (˜300 fold) improvement over the previously developed KUN replicon packaging system employing cytopathic SFV replicon RNA for transient expression of KUN structural genes (Khromykh et al., 1998, J Virol. 72 5967-5977; Varnavski & Khromykh. 1999, supra) and makes feasible large scale commercial production of KUN replicon VLPs for future vaccine and gene therapy applications. The utility of the high titre KUN replicon VLPs produced in packaging cells for vaccine applications was demonstrated by generation in immunized mice of potent CD8+ T cell responses to an encoded immunogen from respiratory syncytial virus. In addition, tetKUNCprME cells were able to package dengue virus replicons into secreted infectious VLPs indicating a possible application of tetKUNCprME cells for production of VLPs encapsidating replicons from distantly related flaviviruses.

The inducible packaging construct of the invention overcomes the problem of apparent cytotoxicity of the structural proteins. Furthermore, in view of the intended uses of KUN replicon VLPs including vaccine and/or protein production applications, the inducible packaging system of the invention avoids the presence of antibiotic in VLP preparations.

Approximately 30-fold induction of KUN CprME mRNA transcription and CprME expression was observed in the established tetKUNCprME cell line upon removal of doxycyline, and the amount of KUN structural proteins produced in tetKUNCprME cells upon induction of expression was sufficient to obtain high titres of secreted infectious VLPs after transfection of KUN replicon RNA. Titres of up to ˜4×10⁸ VLPs per ml were obtained, a yield equal or higher than the viral titres obtained at the peak of wild type KUN virus infection in BHK cells Khromykh & Westaway, 1994, J Virol. 68 4580-4588).

Importantly, the most sensitive method for detection of KUN virus by intracranial injection of suckling mice clearly showed no infectious KUN virus present in VLP preparations from tetKUNCprME cells. In comparison, a BHK packaging cell line expressing a Sindbis virus structural protein cassette produced 1-5×10⁸ of Sindbis or SFV replicon VLPs per ml (Polo et al., 1999, supra). These alphavirus replicon VLP preparations however, contained ˜10⁵ pfu per ml of infectious viruses generated by recombination. Splitting the structural proteins into two expression cassettes in the packaging cell line appeared to remove contamination of these alphavirus replicon VLP preparations with infectious viruses to an undetectable level, but at the same time reduced the titres of replicon VLPs to 5×10⁶-1×10⁷ VLPs per ml (Polo et al., 1999, supra).

Packaging of DEN2 replicon RNAs into secreted VLPs was also achieved in tetKUNCprME cells.

To illustrate the utility of high titre KUN replicon VLPs for vaccination, two VLPs were tested in different mouse strains. Previous studies showed that KUN replicon VLPs injected at doses up to 10⁶ IU per mouse were efficient in induction of immune responses able to protect animals from experimental viral and tumour challenges (Anraku et al., 2002, supra). Using VLPs produced in the new packaging cell line, a dose response for KUN-Mpt VLP was demonstrated in C57BL/6 mice for SIINFEKL-specific CD8 T cells, with increasing doses of VLPs resulting in increased number of induced CD8 T cells. Furthermore, immunisation of BALB/c mice with a single inoculation of 2.5×10⁷ IU of tetKUNCprME-derived KUN replicon VLPs encoding the RSV M2 gene, resulted in the induction of 1400 SYIGSINNI-specific CD8 T cells per-10⁶ splenocytes as measured by ex vivo IFNγ ELISPOT assay and >45% lysis at effector to target cells ratios of 2:1 in chromium release assays.

In summary, the present invention provides a packaging system allowing production of large amounts of high titre secreted KUN replicon virus like particles free of infectious virus and demonstrated that immunization with these particles induced a potent immune response to the encoded immunogen. The packaging cell line thus should prove to be useful for the manufacture of KUN replicon-based vaccines. In addition, the packaging cell line was also capable of packaging other flavivirus replicons and should prove to be useful in basic studies on flavivirus RNA packaging and virus assembly and in the development of gene expression systems based on different flavivirus replicons.

Throughout this specification the aim has been to describe the preferred embodiments of the invention without limiting the invention to any one embodiment or specific collection of features. It will therefore be appreciated by those of skill in the art that, in light of the instant disclosure, various modifications and changes can be made in the particular embodiments exemplified without departing from the scope of the present invention.

The disclosure of each patent and scientific document, computer program and algorithm referred to in this specification is incorporated herein by reference in its entirety.

TABLE 1
Packaging efficiencies of different tetKUNCprME cell clones.

| | VLP titre* |
|---|---|
| Cell Clone | (IU/ml) |
| A3 | 5.7 × 10⁵ |
| A8 | 2.1 × 10⁸ |
| E1 | 2 × 10⁷ |
| E5 | 5.3 × 10⁴ |

*2 × 10⁶ cells were electroporated with ˜15 ug of KUN replicon RNA, RNALeu, and the titres of secreted VLPs harvested at 53 h after electroporation were determined by titration on Vero cells.

TABLE 2
Effect of CprME expression induction time on VLP
production.

| | |
|---|---|
| | VLP production (IU/ml) at hours |
| Time of | post electroporation |

| induction^{a} | 53 h | 68 h |
|---|---|---|
| 0 h | 2.1 × 10⁸ | 3 × 10⁷ |
| 16 h | <100 | 2.9 × 10⁶ |
| 30 h | <100 | 5 × 10⁵ |

*The induction of CprME expression was initiated by removal of doxycycline at indicated times after electroporation with RNAleu RNA.

TABLE 3
Production of secreted KUN replicon VLPs encoding different heterologous genes in the tetKUNCprME
packaging-cell line.

| | | | | | | | | Total VLP |
|---|---|---|---|---|---|---|---|---|
| | | | | | | | | production |
| | VLP Titre (IU/ml) | | | | | | | per 3 × 10⁶ |
| VLP Type | 2 d | 3 d | 4 d | 5 d | 6 d | 8 d | 10 d | cells |
| RNAleuMPt^{a} | 3.1 × 10⁷ | 5.5 × 10⁷ | 3.8 × 10⁸ | — | 2.9 × 10⁸ | 1.3 × 10⁸ | — | 6.5 × 10⁹ |
| KUNgag^{a} | 1 × 10⁷ | 3.9 × 10⁷ | 1.2 × 10⁸ | 1.6 × 10⁷ | — | — | — | 9.5 × 10⁸ |
| RNAleu^{a} | 1.8 × 10⁸ | 1.9 × 10⁸ | — | 2.5 × 10⁶ | — | — | — | 1.6 × 10⁹ |
| repGFP^{b} | 1.6 × 10⁸ | 2.6 × 10⁸ | 3.7 × 10⁸ | 2 × 10⁸ | — | — | — | 5.2 × 10⁹ |
| repPAC^{c} | — | — | 1.6 × 10⁸ | — | 2.2 × 10⁸ | — | 1.9 × 10⁸ | 6.5 × 10⁹ |
| repPACβ-gal^{d} exp 1 | 4 × 10⁵ | — | 1.1 × 10⁸ | — | 2.3 × 10⁸ | — | — | 3.4 × 10⁹ |
| repPACβ-gal^{e} exp 2 | 1.2 × 10⁶ | — | 1.6 × 10⁹ | — | 1.1 × 10⁹ | — | — | 5.4 × 10¹⁰ |
| repPACβ-gal^{e} exp 3 | 5 × 10⁶ | — | 1.3 × 10⁸ | — | 1.8 × 10⁸ | 3.3 × 10⁸ | — | 1.3 × 10¹⁰ |

^{a-d}3 × 10⁶ cells were electroporated with ˜2O μg RNA, seeded onto one 10 cm culture dish, and incubated in different volumes of medium and for different times prior to harvesting VLPs. 6 ml^{a}, 5 ml^{b}, 10 ml^{d} or of medium in each dish were used for initial VLP harvest and to replace harvested VLPs to allow further VLP production and harvests.
^{C}10 ml of medium were harvested at days 4 and 6, and 15 ml of medium were harvested at day 10.
^{e}3 × 10⁶ electroporated cells were seeded onto two 10 cm culture dishes, and cells in each dish were incubated in 10 ml of medium that was replaced with 10 ml of fresh medium at each indicated harvest day. Total VLP production was calculated by combining amounts of VLPs obtained in each harvest.
“—” shows that VLPs were not harvested at this time and the medium remained unchanged until the next harvest.

TABLE 4
Packaging efficiencies (pfu/ml) of KUN replicon RNAs with
different cell-adaptive mutations into VLPs in tetKUNCprME
packaging cells

| | Day2 | Day4 | Day6 | Day8 |
|---|---|---|---|---|
| Wt | 5 × 10⁶ | 1.3 × 10⁸ | 1.8 × 10⁸ | 3.3 × 10⁸ |
| NS2A/A30P | 4 × 10⁶ | 1 × 10⁸ | 3.4 × 10⁸ | 6 × 10⁸ |
| NS2A/N101D | 4.4 × 10⁴ | 1.3 × 10⁷ | 4 × 10⁷ | n.d. |
| NS5/P270S | 1.0 × 10⁵ | 6 × 10⁷ | 1.3 × 10⁸ | n.d. |
| NS2A/A30P/N101D | 1 × 10⁴ | 1.1 × 10⁵ | 1.2 × 10⁶ | 9 × 10⁶ |

1. A packaging construct for regulatable expression of flavivirus structural proteins in an animal cell, said vector comprising a regulatable promoter operably linked to a nucleotide sequence encoding a flavivirus structural protein translation product that comprises C protein, prM protein and E protein.

2. The packaging construct of claim 1, wherein the regulatable promoter is tetracycline-repressible.

3. The packaging construct of claim 2 wherein the regulatable promoter is a tetracycline repressible CMV promoter.

4. The packaging construct of claim 1, wherein the nucleotide sequence encodes one or more variant or mutated flavivirus structural proteins respectively having at least 80% amino acid sequence identity to C protein, prM protein or E protein.

5. The packaging construct of claim 1, further comprising an IRESNeo selection marker nucleotide sequence.

6. The packaging construct of claim 1 wherein the C protein, prM protein and E protein are structural proteins of Kunjin virus.

7. A packaging cell comprising the packaging construct of claim 1.

8. A packaging cell comprising the packaging construct of claim 2 and a tetracycline transactivator construct.

9. The packaging cell of claim 7, which is a BHK21 cell.

10. A flaviviral packaging system comprising:
(i) a packaging construct according to claim 1; and
(ii) a flaviviral expression construct comprising:
(a) a flaviviral replicon;
(b) a heterologous nucleic acid; and
(c) a promoter operably linked to said replicon.

11. The flaviviral packaging system of claim 10, wherein the flaviviral replicon is a Kunjin virus replicon, Dengue virus replicon or a West Nile virus replicon.

12. The flaviviral packaging system of claim 10, wherein the heterologous nucleic acid encodes one or more proteins expressible in an animal cell.

13. The flaviviral packaging system of claim 12, wherein the one or more proteins is/are immunogenic.

14. The flaviviral packaging system of claim 10 wherein the replicon encodes on or more one or more mutated structural proteins.

15. The flaviviral packaging system of claim 14 wherein the mutated structural protein comprises a mutation selected from the group consisting of:
(i) Leucine residue 250 substituted by Proline in the NS 1 nonstructural protein.
(ii) Alanine 30 substituted by Proline in the nonstructural protein NS2A;
(iii) Asparagine 101 substituted by Aspartate in the nonstructural protein NS2A; and
(iv) Proline 270 substituted by Serine in the nonstructural protein NS5.

16. The flaviviral packaging system of claim 10, wherein the regulatable promoter is tetracycline-repressible.

17. The flaviviral packaging system of claim 16 wherein the regulatable promoter is a tetracycline repressible CMV promoter.

18. The flaviviral packaging system of claim 10 wherein the flaviviral expression construct is in RNA form.

19. A packaging cell comprising the flaviviral packaging system of claim 10.

20. A packaging cell comprising the flaviviral packaging system of claim 16 and a tetracycline transactivator construct.

21. The packaging cell of claim 19 or claim 20, which is a BHK21 cell.

22. A method of producing flavivirus VLPs including the step of:
(i) introducing the packaging construct of claim 1 into a host cell to thereby produce a packaging cell;
(ii) introducing into said packaging cell a flaviviral expression construct comprising:
(a) a flaviviral replicon;
(b) a heterologous nucleic acid; and
(c) a promoter operably linked to said replicon; and
(iii) inducing production of one or more VLPs by said packaging cell.

23. The method of claim 22, wherein the flaviviral expression construct is in RNA form.

24. Flaviviral VLPs produced according to the method of claim 22.

25. An immunotherapeutic composition comprising the VLPs of claim 24 and a pharmaceutically acceptable carrier diluent or excipient.

26. The immunotherapeutic composition of claim 25, which is a vaccine.

27. A method of producing a recombinant protein including the step of infecting a host cell with the VLPs of claim 24, whereby said heterologous nucleic acid encoding said protein is expressed in said host cell.

28. The method of claim 27, wherein the host cell is a mammalian cell.

29. A method of immunizing an animal including the step of administering the immunotherapeutic composition of claim 26 to the animal to thereby induce an immune response in the animal.

30. The method of claim 29, wherein the animal is a mammal.

31. The method of claim 30, wherein the mammal is a human.
